(19) United States
(12) Patent Application Publication
(10) Pub. No.: US 2013/0280317 A1
(43) Pub. Date: Oct. 24, 2013

(54) COATED NUTRACEUTICAL AND PHARMACEUTICAL COMPOSITIONS

(71) Applicant: Jan Erik Olsen, Tromso (NO)

(72) Inventor: Jan Erik Olsen, Tromso (NO)

(73) Assignee: AYANDA GROUP AS, Tromso (NO)

(21) Appl. No.: 13/697,429

(22) PCT Filed: May 13, 2011
(86) PCT No.: PCT/GB2011/050923

§ 371 Date: Jul. 11, 2013

(30) Foreign Application Priority Data
May 13, 2010 (GB) 1008049.7

(51) Int. Cl. A61K 9/68

(52) U.S. Cl. CPC A61K 9/0058
USPC 424/440

(57) ABSTRACT

This invention provides oral nutraceutical or pharmaceutical compositions comprising a physiologically tolerable capsule provided with a chewing or bubble gum coat (preferably a chewing gum coat) and containing a physiologically tolerable lipid.

This invention relates to nutraceutical and pharmaceutical compositions in the form of a physiologically tolerable capsule provided with a chewing or bubble gum coat and containing a physiologically tolerable lipid, especially a fatty acid ester oil, and optionally a drug substance, particularly a drug of abuse, especially a stimulant, sedative, tranquiliser, strong pain reliever (e.g. an opioid), or a psychoactive agent.

The term fatty acid ester oil is used herein to relate to acyl glycerides and phospholipids, i.e. compounds comprising a fatty acid side chain linked by an ester group to an “alcohol” (e.g. polyol) residue. Such compounds are important dietary sources of fatty acids, in particular polyunsaturated fatty acids (PUFAs) and more especially the essential fatty acids. They may also serve as sources for dietary replacements of essential fatty acids, e.g. of conjugated linoleic acid (CLA) which may be used in a weight reduction diet. Particularly important essential fatty acids include the ω-3, ω-6 and ω-9 acids such as eicosapentaenoic acid (EPA) and docosahexaenoic acid (DHA). Other fatty acids commonly used in nutraceuticals and pharmaceuticals include arachidonic acid (AA), alpha linolenic acid (ALA), conjugated linolenic acid (CLN), dihomo-gamma linolenic acid (DGLA) and gamma linolenic acid (GLA). Such fatty acids typically will contain 12 to 26 carbons, more typically 16 to 22 carbons, and will have a saturated or mono- or poly-ethylenically-unsaturated hydrocarbyl chain.

Typical dietary sources of such fatty acid ester oils include lipids such as animal, fish, plant or microorganism triglycerides and phospholipids, especially the triglycerides. Mono or diglycerides however can equally be used as can other esters, e.g. lower alkyl (e.g. C₁₋₆ alkyl, for example ethyl) esters as well as free fatty acids or physiologically acceptable salts thereof and fatty acid ester waxes. Particularly important sources are fish oils, in particular oily fish oils such as cod-liver oil, halibut-liver oil, etc. as these are rich in ω-3, ω-6 and ω-9 fatty acids.

However, as anyone who, in childhood, has been on the receiving end of fish oils will recall, the taste, mouthfeel and smell can be vile. In part this is due to the sensitivity to oxidation of the fish oil. As a result fatty acid ester oils tend to be administered in capsule form, containing liquid oil within a soft gel casing. Such capsule casings are usually made from mammalian gelatin, typically of porcine or bovine origin. In order to deliver a reasonable dose of the oil, the capsules tend to be rather large and as a result, ingestion often involves the capsule being chewed and bursting in the mouth releasing the unpleasantly tasting oil contents.

There is thus a continuing need for improved oral administration forms for fatty acid ester oils.

Many drugs which are prescribed for a legitimate use are misused or abused. Three types of drugs are particularly prone to abuse: opioids, CNS depressants, and stimulants. Examples include morphine, morphine-6-glucuronide, diamorphine, hydrocodone, oxycodone, methadone, codeine, diphenoxilate, propoxyphene, dextropropoxyphene, oxymorphone, pentazocine, levorphanol, hydromorphone, buprenorfine, ketobemidone, pethidine, meperidine, oxycodone, fentanyl, tramadol, tapentadol, levorphanol, butorphanol, benzodiazepines (e.g. alprazolam, diazepham), zolpidem, methylphenidate, amphetamines, barbiturates, and pentobarbital.

Such prescription drugs may for example become available for abuse by being stolen from or sold by the legitimate patient. In order to maximise sales or to present the drug in a form suitable for snorting or injection, such drugs are frequently crushed, and optionally diluted and re-tableted or solvent extracted.

A number of strategies have been developed to hinder or prevent such dilution or subsequent abuse. One for example involves including in opioid oral dosage forms an opioid anti-agonist, for example naloxone, which does not block the opioid activity when the oral dosage form is consumed but which will be extracted with the opioid on solvent extraction and will then block the opioid's effect on injection of the extract. A further strategy is to present the drug substance in an inactive pro-drug form, e.g. an enol ester, which requires digestive enzymes to release the active drug. In this case the prodrug is inactive if snorted as a powder or injected following extraction. Other strategies involve incorporating an irritant (e.g. capsaicin) or a bitter component (e.g. denatonium benzoate) to limit snorting or injection abuse.

Still further strategies involve presentation in a hard, not easily crushable dosage form or in a form which gels on addition of water or attempted crushing.

Such strategies however may risk reducing patient acceptability of the oral dosage form when consumed by the legitimate recipient and there is thus a continuing need for abuse-deterring oral dosage forms for drugs subject to abuse.

We have now found that the unpleasant effects of bursting fish oil capsules on ingestion may be overcome by providing the capsules with a shell of chewing or bubble gum, the taste of which serves to mask the taste of the fish oil. Perceived as a confectionery product, rather than as “something healthy”, these nutraceutical compositions have increased acceptability to juvenile recipients. Moreover, drugs subject to abuse may be presented in a legitimate user friendly but abuse-deterring form by including the drug substance in physiologically tolerable lipid, e.g. a fish oil, contained within a chewing or bubble gum coated capsule. Any attempt to extract the drug of abuse will be hampered if not prevented outright. Thus crushing is difficult and even if crushing is effected under cryogenic conditions, the resulting fragments are not a free flowing powder when returned to ambient temperatures. Solvent extraction is complicated since the extracts will be contaminated by components from the lipid, the capsule and the chewing or bubble gum coat. Moreover, if the drugs are presented in an lipid which contains unsaturated fatty acids (e.g. omega-3, omega-6 or omega-9 fatty acids), for example fish oils, any attempt to crush or to solvent extract the drug of abuse will result in an evil-smelling and tasting product which will be unattractive to abusive users. This arises from the susceptibility of such oils to oxidation. The chewing or bubble gum coated capsules however are readily consumed by the legitimate user with no unpleasant effects.

As an alternative to drugs of abuse, the pharmaceutical compositions according to the invention may conveniently contain drug substances effective in or taken up from within the mouth, e.g. drugs for the treatment of diseases of the mouth, since the act of chewing causes the drug substance to be retained within the mouth for a time sufficient for the drug to take effect. Moreover, by virtue of the chewing of the compositions, buccal uptake and subsequent further uptake from the gastrointestinal tract will provide both an immediate and a sustained uptake for the drug substance. The buccal uptake furthermore will increase the efficiency of drug substance uptake by virtue of reducing the first pass effect.

Thus viewed from one aspect the invention provides an oral nutraceutical or pharmaceutical composition comprising a physiologically tolerable capsule provided with a chewing or bubble gum coat (preferably a chewing gum coat) and containing a physiologically tolerable lipid.

In a preferred embodiment, the lipid in the compositions according to the invention comprises a fatty acid ester, particularly a polyunsaturated fatty acid ester, more especially an omega-3 acid ester. The lipid preferably comprises, consists essentially of or is a marine oil, e.g. a fish, shellfish or marine cephalopod oil.

Examples of omega-3 acids include a-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of omega-6 acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of omega-9 acids include oleic acid, eicosenoic acid, mead acid, erucic acid and nervonic acid.

Where the compositions of the invention are nutraceuticals, they may if desired contain vitamins and/or minerals; however they are preferably otherwise substantially free of drug substances.

Where the compositions of the invention are pharmaceuticals, they will contain a drug substance dissolved and/or dispersed in the lipid. Dispersion may be of a solid or of a discontinuous liquid phase, e.g. an aqeous phase. If desired, the drug substance may be present in delayed or sustained release form. This may be achieved by conventional microencapsulation and dispersion of the encapsulated drug in the lipid phase or in a discontinuous liquid phase.

The drug substance is preferably a drug of abuse; however other drugs may be used, for example antibiotics, decongestants and antitussives.

By drug of abuse is meant a drug substance or combination of drugs having a legitimate use selected from the group consisting of stimulants, sedatives, tranquilizers, strong pain relievers, and psychoactive agents. By strong pain reliever is meant drugs such as opioids, morphine, codeine, oxycodone, hydrocodone, diamorphine, pethidine, tramadol, buprenorphine, venlafaxine, nefopam, carbamazepine, gabapentin and pregabalin and tricyclic antidepressants such as amitriptyline, but not over-the-counter available analgesics such as acetyl salicylic acid, paracetamol, ibuprofen and other NSAIDs (however some doses and combinations of over the counter drugs may require prescription in certain jurisdictions and such doses/combinations are considered drugs of abuse).

Examples of drugs of abuse include codeine, morphine (and morphine derivatives), hydrocodone, oxycodone, diamorphine, pethidine, tramadol, buprenorphine, propoxyphene, hydromorphone, meperidine, diphenoxylate, barbiturates (e.g. pentobarbital sodium), benzodiazepines (e.g. diazepam, alprazolam and flunitrazepam), amphetamines (e.g. amphetamine, dextroamphetamine, klysine-d-amphetamine), methyl phenidate, zolpidem, methadone, mephedrone, tetrahydrocannabinol, ketamine, clonidine, mexiletine, tapentadol, and others mentioned above. Antitussives and decongestants are also subject to abuse and are therefore also included. Also included are prescription drugs which contain components that themselves are available over the counter (e.g. drugs such as NSAIDs, aspirin, paracetamol and ibuprofen are usually available over the counter but may also be included in prescription-only analgesics). That is, drug combinations that are prescription-only, e.g. Vicodin, are considered drugs of abuse regardless of whether they contain some over the counter drugs. Further drugs of abuse are listed for example in WO 2005/123039.

If desired, the drug of abuse may be present in the compositions of the invention in prodrug form, e.g. as an ester, which is activated following oral ingestion.

Also if desired, the compositions of the invention may contain an antagonist to the drug substance, i.e. an agent which on injection will block the uptake of the drug of abuse, for example naloxone where the drug of abuse is an opioid. Preferably such antagonists are ones which are inactive following oral administration.

In the pharmaceutical compositions of the invention, the drug substance will typically be present at 10 to 100%, especially 50 to 100% of the dose in conventional oral compositions such as tablets or capsules. These dosages are well known for these drugs and need not be discussed further here.

The capsule shell in the compositions of the invention may be in any convenient form, but preferably it is a gel capsule, particularly a soft gel capsule, for example of gelatin or another suitable hydrocolloid. The loading of liquids into capsules is well known within the pharmaceutical and nutraceutical industries and need not be described further. One particularly suitable soft gel capsule form is described in WO2009/095670.

Viewed from a further aspect the invention provides a method of treatment of a mammalian subject (either human or non-human) by oral administration to said subject of an effective amount of a drug substance, the improvement comprising administering said drug substance in a physiologically tolerable capsule provided with a chewing or bubble gum coat and containing a physiologically tolerable lipid and said drug substance.

Viewed from a further aspect the invention provides a pharmaceutical composition comprising a physiologically tolerable capsule provided with a chewing or bubble gum coat and containing a physiologically tolerable lipid and a drug substance, for use in medicine.

Viewed from a further aspect the invention provides a pharmaceutical composition comprising a physiologically tolerable capsule provided with a chewing or bubble gum coat and containing a physiologically tolerable lipid and a drug of abuse, for use in treatment by oral administration of a condition responsive to said drug of abuse.

Viewed from a still further aspect the invention provides the use of a drug substance for the manufacture of a medicament according to the invention for use by oral administration in the treatment of a condition responsive to said drug substance.

Besides drug substances, the compositions of the invention may contain further components such as nutrients, e.g. lipids, (especially triglycerides and phospholipids, typically of plant or marine animal origin), vitamins, minerals, and folic acid, pH modifiers, viscosity modifiers, flavours, aromas, sweeteners, colorants, antioxidants, etc. Preferably however the compositions are substantially free of cariogenic sugars.

It is particularly preferred that the compositions according to the invention contain a citrus flavour (e.g. orange or lemon oil) in order to mask any remaining oil taste on chewing. It is also particularly preferred that the compositions according to the invention contain xylitol, e.g. as 0.5 to 50% wt., preferably 1 to 40 % wt., e.g. 15 to 40% wt., in order to mask both taste and mouth feel. These may be in the capsule contents or in the chewing or bubble gum coat or both.

The compositions of the invention will be in dose unit form, preferably with each dose unit having a weight of 500 to 3000 mg, especially 1000 to 2500 mg, particularly 1200 to 2000 mg.

The compositions of the invention may if desired be provided with a further coating, e.g. a wax or sugar coating, outside the chewing or bubble gum coat. This may be applied in conventional fashion.

The lipid in the pharmaceutical compositions of the invention may also contain solubilisers in order to increase the solubility of the drug substance in the oil phase. Suitable solubilisers would be known to a person skilled in the art and include Chremophor EL™, castor oil, Tween 80™, Solutol™, Lutrol™ and Olestra. Likewise the drug substance may be complexed with cyclodextrin to enhance its dispersibility.

Other than any drug substance, fatty acid esters may form part or the whole of the lipid phase, preferably at least 10% wt, more especially at least 50% wt, particularly at least 80% wt. of that phase. They may be used as single compounds or as compound mixtures, e.g. plant or marine oils.

Besides prescription drugs, several drugs available over-the-counter have been subject to abuse, e.g. anti-tussives, decongestants, and the like. Prescription anti-tussives and decongestants may also be subject to abuse. The present invention is also applicable to such drug substances (including doses and combinations of over the counter drugs such as NSAID's, paracetamol, aspirin and ibuprofen which may only be available by prescription in certain jurisdictions).

Examples of over the counter drugs, including anti-tussives and decongestants that may be used include: dextromethorphan and several of the opoids listed above, pseudoephedrine; phenylephrine; phenylpropanolamine; and dextromethorphan; optionally in combination with guaifenesin and/or analgesics such as aspirin, ibuprofen and other NSAIDs.

Examples of drugs suitable for buccal uptake using the compositions of the invention include: buprenorphine; midazolam; carvedilol; propafenone; acyclovir; propranolol; insulin; alpha-interferon; octreotide; leutinising hormone releasing hormone; and triamcinolone acetonide. For buccal uptake, it is preferred that the compositions include a permeation enhancer, e.g. cyclodextrin, menthol, or azone. This may be in the capsule or the gum coating. It is also preferred to include a mucoadhesive in the gum coating.

As mentioned, the filled capsule centres of the compositions according to the invention may be prepared in conventional fashion. Commercially available capsules, e.g. soft gelatin capsules filled with fish oil, may even be used. These filled capsules may then be provided with a chewing or bubble gum coat, again in conventional fashion, e.g. as described in US-B-6551634.

Chewing gum mass, used to coat the capsules in the present invention is generally a mixture of a gum base (e.g. a synthetic rubber), softener (e.g. glycerine), sweetener (e.g. xylitol), and flavour (e.g. mint). The mass will generally be used in powder form.

In a particularly preferred embodiment, the compositions are prepared by (a) pregumming the capsules with a gum solution, e.g. gum arabic dissolved in a polyol (e.g. maltitol syrup), (b) drying, (c) adding a liquid and powdered chewing gum/bubble gum mass, (d) drying, (e) repeating steps c and d until the desired gum coating is achieved, generally about 20 times to achieve an about 1 g coat, (f) hard coating with an aqueous solution containing crystalline sugars and polyols (e.g. maltitol) and evaporating off the water, and (g) polishing and glazing with edible wax. Gum coating may be effected at ambient temperature, hard coating will generally be effected at 30-35° C. The powdered gum mass may be any suitable chewing or bubble gum base, for example the product available as Chewycoat (trade mark) from Alsiano, Denmark.

The pH (at ambient temperature) of the gummy coating mass of the compositions according to the invention and/or of the pregumming solution used to adhere the gummy coating in the compositions according to the invention may be lowered to less than 4.5 but still above 1, preferably to pH<4, especially 2-3.8, e.g. 2.5-3.6. We have surprisingly found that lowering the pH of the gummy coating mass and/or pregumming solution results in a gradual softening of the capsule shell which avoids the presence of solid residues (e.g. solid gelatin residues) on chewing and therefore results in a more pleasant chewing experience.

We have also found that, although the capsule shell may be required in order to give the dosage form the necessary stability to undergo the tough treatment of coating, after the coating process the capsule shell barrier is no longer necessary since the coating layers provide enough structural stability.

The invention will now be illustrated further with reference to the following non-limiting Examples.

### EXAMPLE 1

### Fish Oil Tablets

Commercially available 6 Oval soft gelatin capsules containing fish oil were pregummed with a polyol solution of gum arabic (e.g. a solution in maltitol syrup) before being covered with Gummycoat (trade mark) dispersed in liquid polyol and dried twenty times. The gum coated capsules were provided with a hard sugar coating by rotation in an aqueous sugar (e.g. crystalline maltitol) dispersion at 30-35° C. to evaporate off the water. The coated capsules were then talc and wax polished. On being chewed, the tablets had no discernible fishy taste.

### EXAMPLE 2

### Methylphenidate Tablets

10 mg of methylphenidate in 600 mg fish oil (e.g. cod liver oil) is loaded into a soft gelatin capsule which is then coated as in Example 1.

### EXAMPLE 3

### Amphetamine Tablets

Example 2 is repeated using 10 mg amphetamine in place of the methylphenidate.

### EXAMPLE 4

### Zolpidem Tablets

Example 2 is repeated using 10 mg zolpidem in place of the methylphenidate.

### EXAMPLE 5

### Methadone Tablets

Example 2 is repeated using 10 mg methadone in place of the methylphenidate.

### EXAMPLE 6

### Phenylephrine Tablets

Example 2 is repeated using 10 mg phenylephrine in place of the methylphenidate.

### EXAMPLE 7

### Ephedrine/Pseudoephedrine Tablets

Example 2 is repeated using 25 mg ephedrine or 60 mg pseudoephedrine in place of the methylphenidate.

### EXAMPLE 8

### Phenylpropanolamine Tablets

Example 2 is repeated using 25 mg phenylpropanolamine in place of the methylphenidate.

### EXAMPLE 9

### Dextromethorphan Tablets

Example 2 is repeated using 15 mg dextromethorphan in place of the methylphenidate.

### EXAMPLE 10

### Noscapine Tablets

Example 2 is repeated using 25 mg noscapine in place of the methylphenidate.

### EXAMPLE 11

### Morphine Tablets

Example 2 is repeated using 5 mg morphine in place of the methylphenidate.

### EXAMPLE 12

### Tramadol Tablets

Example 2 is repeated using 50 mg tramadol in place of the methylphenidate.

### EXAMPLE 13

### Tapentadol Tablets

Example 2 is repeated using 50 mg tapentadol in place of the methylphenidate.

### EXAMPLE 14

### Hydrocodone Tablets

Example 2 is repeated using 5 mg hydrocodone in place of the methylphenidate.

### EXAMPLE 15

### Codeine Tablets

Example 2 is repeated using 30 mg codeine in place of the methylphenidate.

1. An oral nutraceutical or pharmaceutical composition comprising a physiologically tolerable capsule provided with a chewing or bubble gum coat (preferably a chewing gum coat) and containing a physiologically tolerable lipid.

2. A composition as claimed in claim 1 wherein said lipid comprises a fatty acid ester.

3. A composition as claimed in claim 2 wherein said lipid comprises a fish, shellfish or marine cephalopod oil.

4. A pharmaceutical composition as claimed in claim 1 wherein said capsule contains a drug substance.

5. A composition as claimed in claim 4 wherein said drug substance is a drug of abuse.

6. A method of treatment of a mammalian subject (either human or non-human) by oral administration to said subject of an effective amount of a drug substance, the improvement comprising administering said drug substance in a physiologically tolerable capsule provided with a chewing or bubble gum coat and containing a physiologically tolerable lipid and said drug substance.

7. A method as claimed in claim 6 wherein said coated capsule is chewed to allow buccal uptake of said drug substance.

8. A pharmaceutical composition comprising a physiologically tolerable capsule provided with a chewing or bubble gum coat and containing a physiologically tolerable lipid and a drug substance, for use in medicine.

9. A pharmaceutical composition comprising a physiologically tolerable capsule provided with a chewing or bubble gum coat and containing a physiologically tolerable lipid and a drug of abuse, for use in treatment by oral administration of a condition responsive to said drug of abuse.

10. The use of a drug substance for the manufacture of a composition as claimed in claim 4 for use by oral administration in the treatment of a condition responsive to said drug substance.

11. A process for the preparation of a composition as claimed in claim 1 which comprises coating a lipid-containing capsule with a chewing or bubble gum base.
